(19) United States
(12) Patent Application Publication
(10) Pub. No.: US 2012/0003726 A1
(43) Pub. Date: Jan. 5, 2012

(54) APPARATUS AND METHOD FOR CALIBRATION OF NON-CONTACT THERMAL SENSORS

(75) Inventors: Ross Peter Jones, Cambridge (GB); David James Squirrell, Wiltshire (GB)

(73) Assignee: ENIGMA DIAGNOSTICS LIMITED, Wiltshire (GB)

(21) Appl. No.: 12/673,210

(22) PCT Filed: Aug. 15, 2008
(86) PCT No.: PCT/GB08/02773

§ 371 Date: May 6, 2011

(30) Foreign Application Priority Data
Aug. 15, 2007 (GB) 0715854.6

(51) Int. Cl. C12M 1/40; G01K 15/00; G01N 25/00

(52) U.S. Cl. 435/286.1; 374/45; 374/1; 422/62; 374/E15.001

(57) ABSTRACT

Biochemical assay apparatus uses a container with a sleeve of electrically-conductive material (300) to heat it. The heating is done inside a chamber and a contactless heat sensor (110) such as a thermopile or a bolometer, also inside the chamber, is used to monitor the temperature of the electrically conductive material (300). There are many factors that distort the output of the heat sensor (110), particularly as the temperature rises and properties such as emissivity change, or as time goes by and tarnishing and dust affect the heat sensor output. Because the sleeve has low thermal mass and heat transfer only has to happen over short distances, it is relatively easy to calculate a change in actual temperature of the electrically conductive material (300) when subjected to a known pulse of drive current and this property can be used to calibrate the performance of the heat sensor (110) in situ in the chamber.

The present invention relates to apparatus for, and a method for use in, the calibration of non-contact thermal sensors. It also relates to apparatus for, and a method for use in, checking the correct functioning of a reaction using non-contact thermal sensors. It finds particular application in heating apparatus for biochemical samples, an example being those based on polymerase chain reactions (“PCR”).

It is sometimes important to be able to measure the temperature of a body without touching it. The rate or extent of a biochemical reaction can for example be indicated by its temperature but any contact could change the environment, disturb the reaction or cause contamination.

It is known to measure temperature based on the level of black body radiation given out by a body, in particular in the infrared region of the electromagnetic spectrum. There are three main types of non-contact thermal sensor for this purpose: the thermopile, the bolometer and the pyroelectric sensor. These all respond to the heating effect of received infrared radiation to generate an electrical signal indicative of temperature.

A thermopile is based on thermocouples connected in series. A thermocouple is made of two dissimilar conductors. When the two ends of a thermocouple have a temperature difference, it will generate an output voltage. The thermopile amplifies this by using more than one thermocouple.

A bolometer is based on a thermistor which is a device made from a material that changes its electrical resistance with temperature. In a bolometer, the material is used as a membrane which receives the infrared radiation from an object.

A pyroelectric sensor is based on the property of a pyroelectric crystal that when a pyroelectric crystal is heated (or cooled) the expansion (or contraction) is anisotropic causing the material to be strained, and a voltage is generated across it due to the resulting dipole field.

In practice, an important factor is calibration of the non-contact thermal sensor. The output of the sensor needs to have a known relationship to the temperature being measured. However, many complicating factors may be present. The sensor will have a field of view for receiving the infrared radiation and the object or surface of interest may not fill that field of view. Other features of the sensing environment may contribute higher or lower levels of infrared radiation which affect the reading given by the sensor and the extent to which that happens can be variable with temperature. Over time, tarnishing and dust in the environment and the like can affect emissivity of the surfaces involved and will again affect the reading given by the sensor.

In biochemical apparatus for heating fluid samples and monitoring their temperature, it is known to put the sample in a glass capillary test tube with a coating of electrically conductive polymer (“ECP”). This capillary assembly is mounted inside a generally cuboid block of aluminium and the sample is heated by delivering an electric current to the ECP coating. The ECP coating is black, to maximise its emissivity, and the internal surfaces of the block are polished to minimise the contribution they make to infrared radiation. A heat sensor such as a thermopile is mounted near the ECP so as to monitor its temperature.

In order to calibrate the heat sensor, measurements can be made prior to mounting the capillary assembly and heat sensor in the block. An algorithm based on the actual conditions inside the block can then be used to convert the measurements to those that would be seen when the capillary assembly and sensor are in situ in the block. This algorithm has to take several factors into account, these in many cases varying with actual temperature, and builds in error terms to compensate for, for instance:
temperature of the heat sensor itself
temperature differences between the components of the heat sensor, the aluminium block and the mountings for components within it, and the ECP
geometry and emissivity variations

The algorithmic approach is complicated and has the disadvantage that it cannot easily take account of changes over time, such as tarnishing of surfaces.

In an alternative approach, a heat sensor such as a thermistor can be built into the ECP itself. However, this loses the advantage of a contact-free heat sensor and disturbs heat fluxes in use of the apparatus.

According to a first aspect of the present invention, there is provided apparatus for applying monitored temperature changes to samples held in containers, the apparatus comprising:
i) a chamber for holding one or more containers;
ii) a piece of electrically conductive material for use in heating a container in the chamber;
iii) one or more contactless heat sensors for measuring the temperature of at least a portion of the electrically conductive material when in the chamber;
iv) a drive current source for applying a drive current to the electrically conductive material, when in the chamber, so as to change its temperature; and
v) a control circuit adapted to control the drive current to follow a test sequence for use in one of calibrating the one or more contactless heat sensors, testing thermal control and determining the contents of the container.

The control circuit may receive an input from the one or more contactless heat sensors corresponding to said test sequence. The control circuit may be adapted to compare the input from the one or more contactless heat sensors to a predicted temperature.

In one embodiment, the control circuit comprises a heat sensor calibrator for calibrating the response of at least one of said one or more heat sensors to changes in temperature of the electrically conductive material,

wherein the heat sensor calibrator is adapted to control the drive current to follow a test sequence for use in calibrating the heat sensor.

A test sequence may comprise a sudden change, for instance a step change or pulse in drive current, the purpose being to induce a calculable change in temperature of the electrically conductive material before it loses significant thermal energy by conduction to a sample in the container or by black body radiation. This is facilitated where the electrically conductive material has low thermal mass so that it will react quickly to the test sequence. It has been recognised that this is particularly the case in known biochemical apparatus where samples are heat treated in tubes heated by electrically resistive, conductive sleeves

The apparatus may further comprise a feedback circuit for controlling the drive current to the electrically conductive material in accordance with a calibrated output of the heat sensor, during use of the apparatus with a sample.

The control circuitry or heat sensor calibrator will conveniently comprise a drive current controller for applying the test sequence, a detector for detecting an output of the heat sensor and a correlator for correlating heat sensor outputs with features of the test sequence.

The piece of electrically conductive material may itself provide at least part of a container, or may be provided as a sleeve or other cover which can be brought into close contact with the container.

Embodiments of the invention in its first aspect are more efficient, the more quickly a test sequence can be applied and a meaningful calibration or test of system function (for example thermal control or correct sample present) carried out. To complete a one-off, absolute calibration or system function test, it must be possible to translate the level of drive current to an actual temperature of the electrically conductive material, for instance calculating it from the electrical energy put in and the mass of the electrically conductive material being heated. This is easier to do accurately where the electrically conductive material shows a quick response to changes in drive current, before heat begins to dissipate. As mentioned above, a quick response will be shown where the electrically conductive material has low thermal mass and there is only a short distance over which heat has to be transferred. These conditions are both found in a known type of apparatus supplied by Enigma Diagnostics for carrying out biochemical processes involving temperature change on liquid samples.

The Enigma apparatus allows rapid thermal transitions to be effected in a biochemical sample. It does this by combining the functions of heater and container in a single unit and designing the system so that the thickness of material through which heat must be transferred is minimised. The containers tend towards being one dimensional or two dimensional: long thin tubes or flat thin tubes where “thin” is a dimension of about 1 mm or 2 mm across. The walls of the containers are constructed at least partially in ECP. A drive current to the ECP produces a very quick temperature increase and cooling is provided by a fan-driven air flow.

The containers have a low thermal mass and respond quickly to the applied heating current or the air flow. The temperature of a sample in the container is controlled through a feedback loop using a thermopile or bolometer to measure the surface temperature of the ECP. An algorithm (developed from heat-flow calculations) is used to determine the temperature of the sample as it responds to temperature changes in the ECP. The heating current and the cooling air flow are driven using computer or microprocessor control so that the temperature of the ECP needed to provide a given temperature in the sample can be overdriven to maximise transition rates.

Because the ECP tube has low thermal mass, for instance being not more than 0.5 g in weight or indeed not more than 0.25 g, its temperature responds rapidly and proportionately to electrical energy applied to it. In an embodiment of the present invention, this can be supplied as a test sequence of one or more pulses in the drive current to provide step changes in the ECP temperature which in turn produces stepped responses from the thermopile or bolometer. The ECP can therefore be used in situ to calibrate the thermopile or bolometer and this can be conveniently done without any external measurement device. This therefore provides a very convenient and non-invasive, in-field checking and calibration method.

A particular embodiment of the present invention in its first aspect thus comprises heat treatment apparatus for biochemical samples, wherein at least one container is at least partially constructed out of a polymeric material as the electrically conductive material and a cross section of the outermost surface of the container in the region of the polymeric material has a minimum dimension of not more than 5 mm and more preferably 3 mm or less.

Certain biochemical processes require the detection of light output from the sample. At least a portion of the wall of a container that might be used in such a process is necessarily transparent to the light that is to be detected. In an embodiment of the present invention, this can be achieved by constructing the container as a thin, electrically conductive sleeve into which a glass capillary tube is inserted. The bottom of the tube for example can then be used to irradiate the sample as necessary and/or to detect light coming from the sample.

A biochemical process that embodiments of the invention are particularly suited for is the polymerase chain reaction (“PCR”). This is exploited to generate billions of copies of segments of DNA from a tiny sample, enabling many research and clinical applications such as disease diagnosis. In a PCR process, a sample is repeatedly heated and cooled and the progress of the reaction is monitored, for instance using fluorescence of probes introduced into the sample. PCR-based techniques are normally laboratory based and in the past have involved a heating block to heat and cool samples in test tubes. The use of a heating system as described above has negated the need for a heating block altogether, substituting the ECP-based containers which can be individually heated, which not only speeds up the process but also creates a lighter more portable instrument, able to carry out several assays simultaneously. This in practice greatly widens the field of application of PCR processes, for example for research and testing.

The use of ECP-based containers which can be individually heated and monitored has the advantage that the thermal mass of each tube can be kept low, increasing responsiveness.

Temperature control must be precise and accurate to allow the biochemical reactions in processes such as PCR to work optimally. Calibration of the response of a contactless heat sensor can therefore be critical. The use of external probes in a system as described above is undesirable because the particularly low thermal mass of the ECP-based containers, which is needed to make them responsive, also makes them susceptible to small perturbations. Embodiments of the invention as described above support an intrinsic method of calibrating the feedback control circuitry.

In effect, the feedback control aspect is run in reverse: energy applied to the ECP provides a controlled temperature shift that should generate a certain response in the heat sensor and this can be used to check and adjust the response of the heat sensor.

ECP is not the only suitable material for use as described above in providing heat to a biochemical sample. Other electrically resistive, conductive materials could well be substituted, such as true conductors, doped polyacetylene or polyaniline, or inorganic materials such as indium tin oxide. However, it is preferable that the material should be optically opaque.

The apparatus described above is not only suitable for calibration of the heat sensor but may also be used to check that the system is functioning property. When an amplification reaction, such as PCR, is carried out there are three possible results—a positive result, a negative result and a test failure.

A positive result is determined when the target DNA is detected. However, if no target DNA is detected, the result could either be negative or a test failure.

In order to differentiate between a negative result and a test failure, control DNA is added which uses the same primer as the target DNA but uses a different sequence and a different probe. If the target DNA is not present, the control DNA will still amplify; thus a result with no detected target DNA but with detected control DNA shows that the test has worked but that there is no target DNA present, i.e. a negative result.

If no control DNA is present then the test has failed. Test failure can happen for several reasons, the most common being errors in thermal control, sample processing or inhibitors in the sample.

The present invention can be used to determine whether the first two of these factors, i.e. errors in thermal control or sample processing are responsible for the test failure.

By using the apparatus and method of the present invention, the comparison of the heat sensor output with the expected result can be used to determine whether the thermal control is functioning correctly, for example whether the drive current source or non contact heat sensor are functioning.

In this particular embodiment, the test sequence is for use in checking the response of one or both of the drive current source and one or more non contact heat sensors. The heat sensor controller may be adapted to determine whether the response of at least one of said one or more heat sensors is within an expected range. If so, the thermal control is functioning.

In another particular embodiment, the test sequence is for use in gaining information about the sample, in particular whether a sample is present. In this embodiment, the response from the at least one of the one or more heat sensors is compared with an algorithm or look up table. Due to its specific heat capacity and volume, the aqueous sample (which is made up of mostly water) makes a large contribution to the thermal mass of the system and there will be a measurable difference in temperature measured by the heat sensor depending whether the container contains a sample or not when the test sequence is applied. Furthermore, the specific heat capacity will differ for different samples and the results are sensitive enough to be able to differentiate between different sample content, depending on the measured temperatures.

Thus the apparatus and method of the present invention can be used to determine whether a sample is present in the container and if so, what the sample is (for example using an algorithm or look-up table).

If there are no errors in the thermal control or the sample processing, then there may be inhibitors in the sample, for example humic acid from soil samples or haemoglobin from blood samples. These may be tested for by repeating the process with a more dilute sample.

A PCR reaction has both heating and cooling cycles and cooling is typically provided by air flow. In any of the above described embodiments, an air flow may also be provided in the apparatus, so as to change the temperature of the container when in the chamber. The heat sensor controller may be adapted to also control the cooling air flow to follow a test sequence. This test sequence may comprise a sudden change, for instance a step change or pulse, the purpose being to induce a calculable change in temperature of the container, in the same manner as the drive pulse for the electrically conductive material.

The use of a test sequence of cooling air flow uses the same method as for the method of using a pulse of drive current. However, the results are asymmetrical and different models must be used to describe the relationship between test sequence and temperature of sample. The difference results from the different manner of heating and cooling. The heating is provided by electrically conductive material which heats the container by conduction, whereas the cooling is provided by cooling air flow which cools the electrically conductive material which in turn cools the container.

According to a second aspect of the present invention, there is provided a method of heat treating a sample in a container, the container comprising at least in part at least one electrically conductive wall, the method using one or more contactless heat sensors to sense the temperature of said wall, which method comprises the steps of:

i) applying a drive current to the electrically conductive wall;

ii) varying the drive current according to a predetermined test sequence;

iii) monitoring the response of at least one of the one or more heat sensors to the test sequence;

iv) comparing the monitored response to the predicted temperature of the electrically conductive wall; and

v) using the comparison for one of calibrating the one or more contactless heat sensors, testing thermal control and determining the contents of the container.

According to a third aspect of the present invention, there is provided apparatus for applying monitored temperature changes to samples held in containers, the apparatus comprising:

i) a chamber for holding one or more containers;

ii) a piece of electrically conductive material for use in heating a container in the chamber;

iii) one or more contactless heat sensors for measuring the temperature of at least a portion of the electrically conductive material when in the chamber;

iv) a drive current source for applying a drive current to the electrically conductive material, when in the chamber, so as to change its temperature; and

v) a heat sensor calibrator for calibrating the response of at least one of said one or more heat sensors to changes in temperature of the electrically conductive material, wherein the heat sensor calibrator is adapted to control the drive current to follow a test sequence for use in calibrating the heat sensor.

According to a fourth aspect of the present invention, there is provided a method of heat treating a sample in a container, the container comprising at least in part at least one electrically conductive wall, the method using one or more contactless heat sensors to sense the temperature of said wall, which method comprises the steps of:

i) applying a drive current to the electrically conductive wall;

ii) varying the drive current according to a predetermined calibration sequence;

iii) monitoring the response of at least one of the one or more heat sensors to the calibration sequence;

iv) using the monitored response to calibrate the response of the heat sensor to changes in temperature of the electrically conductive wall; and

v) heat treating the sample, using the calibrated response of the heat sensor in a feedback loop to control the drive current to heat the electrically conductive wall.

The steps of varying the drive current according to a predetermined calibration sequence and monitoring the response of the heat sensor to the calibration sequence may be done with a calibration fluid present in the container rather than a sample.

The step of using the monitored response to calibrate the response of the heat sensor may be done so that a subsequent output of the heat sensor gives an absolute measure of the temperature of the electrically conductive walls. Alternatively, one may only be looking for changes in behaviour of a sample, or from one sample to another, in which case the absolute measure of temperature may not be essential.

Biochemical assay equipment incorporating a heat sensor calibration arrangement will now be described as an embodiment of the present invention, by way of example only, with reference to the accompanying drawings in which:

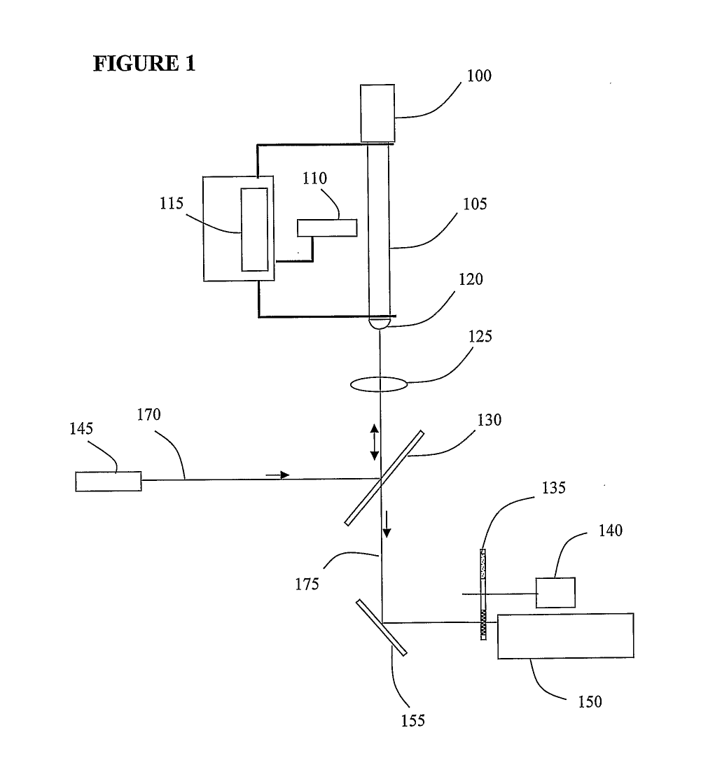

FIG. 1 shows a schematic representation of the equipment in use in a fluorescence-based assay;

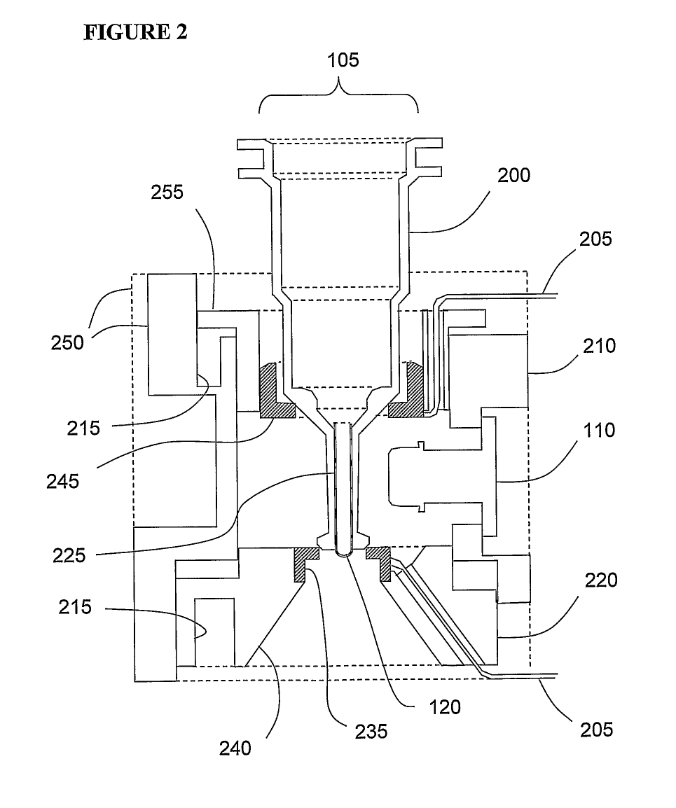

FIG. 2 shows a cross section of a chamber for use in the equipment of FIG. 1, the chamber holding an ECP capillary assembly and including connectors for providing drive current to the ECP material and a contactless heat sensor;

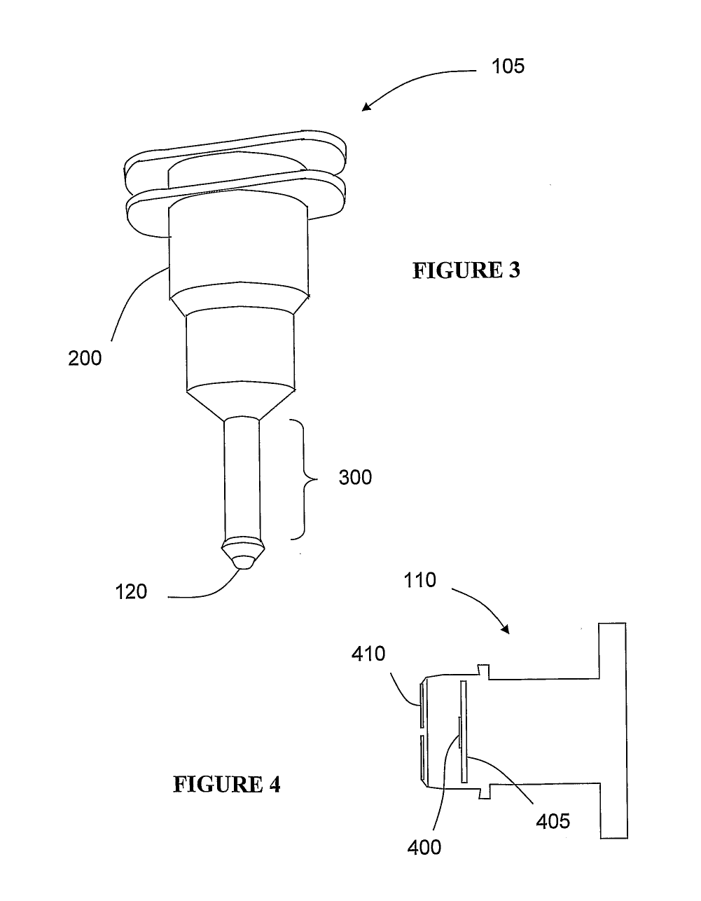

FIG. 3 shows a side elevation, slightly from below, of the ECP capillary assembly of FIG. 2;

FIG. 4 shows in schematic cross section the heat sensor of FIG. 2;

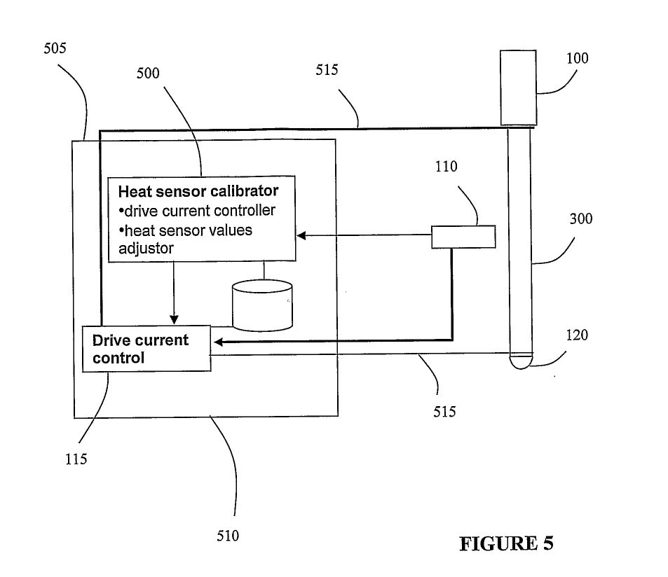

FIG. 5 shows components of a heating circuit for use in the equipment of FIG. 1;

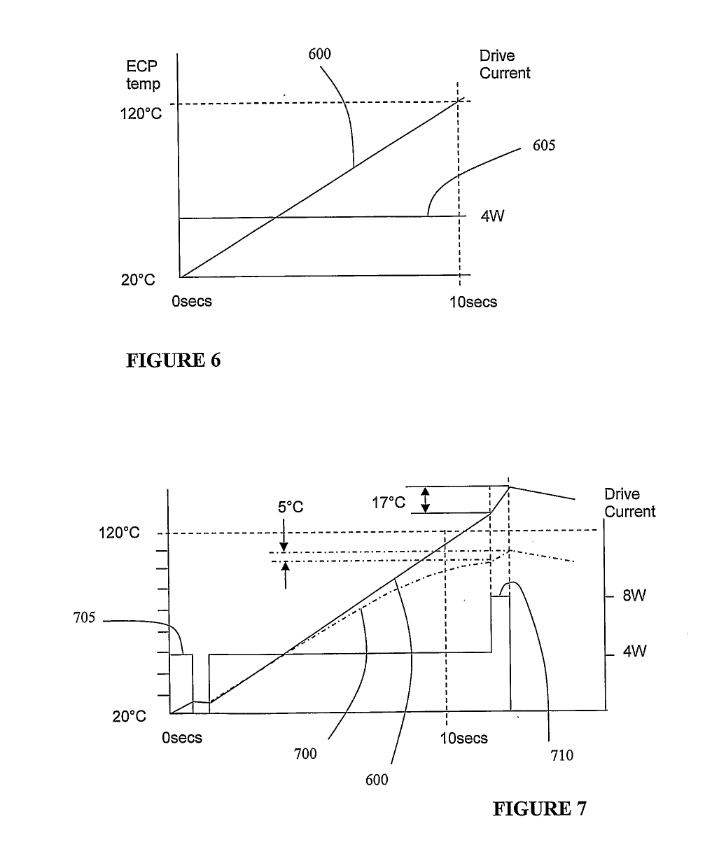

FIG. 6 shows a graph of ECP temperature against time under constant drive current;

FIG. 7 shows a graph of the use of calibration pulses in the drive current to compensate for changes in the chamber and/or heat sensor of FIG. 2 over time;

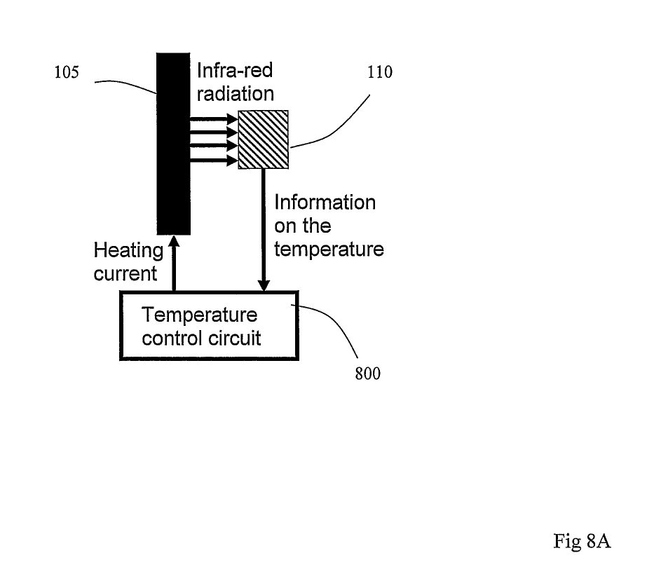

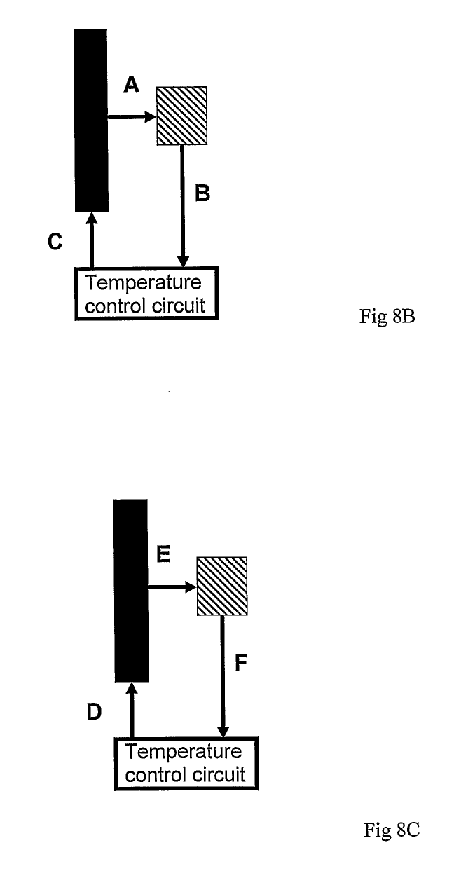

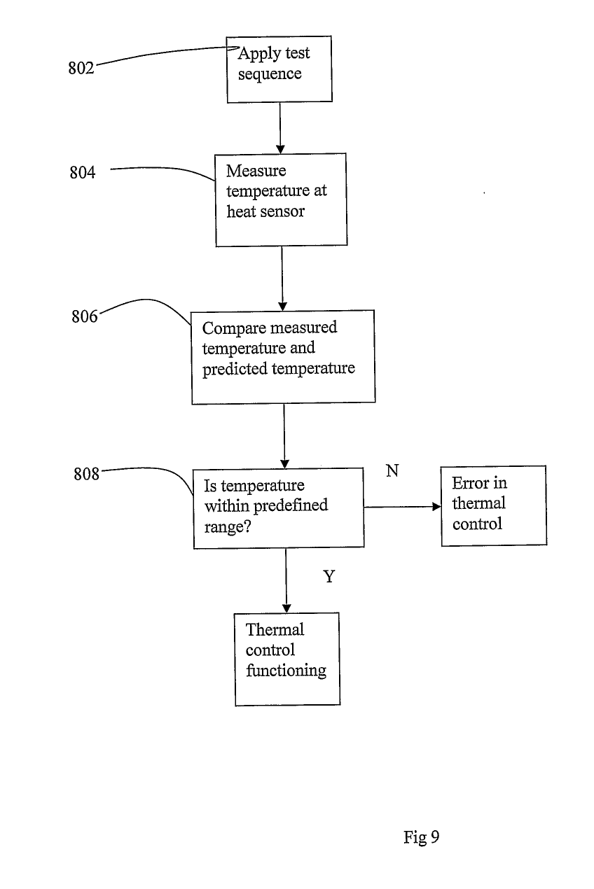

FIGS. 8A-8C are schematic illustrations of the temperature control circuit and the feedback control cycle;

FIG. 9 is a flow diagram illustrating the method for determining testing the thermal control is functioning;

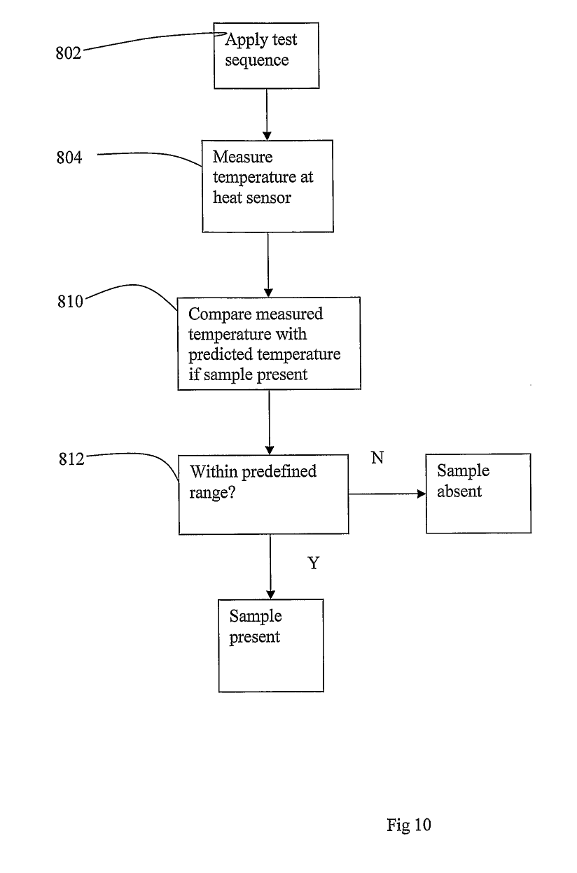

FIG. 10 is a flow diagram illustrating the method for determining testing whether a sample is present;

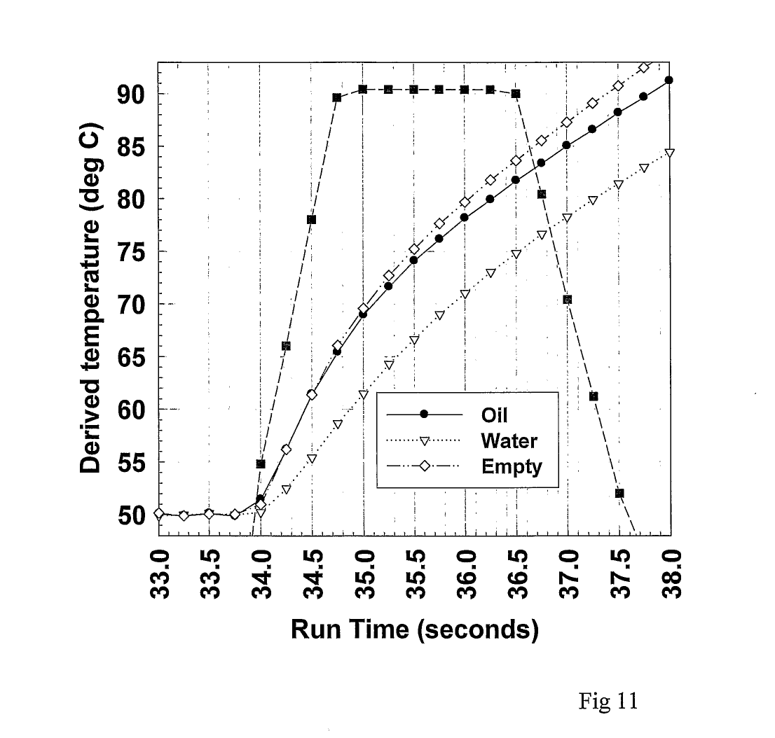

FIG. 11 is a graph showing the effect of a heat pulse on different samples;

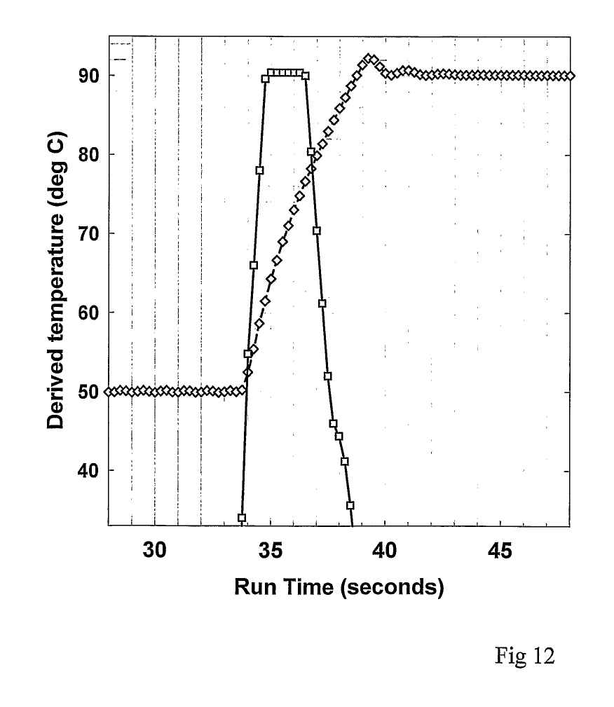

FIG. 12 is a graph showing the temperature of ECP in response to a current pulse;

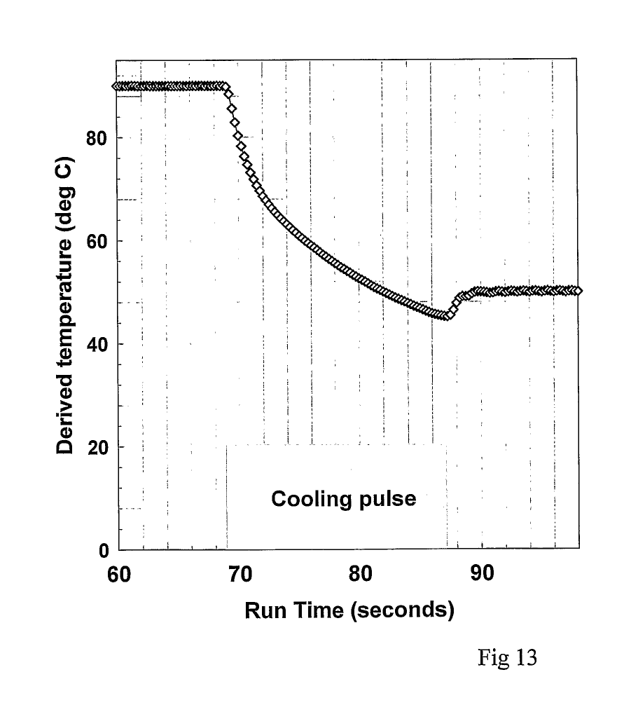

FIG. 13 is a graph showing the temperature of ECP in response to a pulse of cool air; and

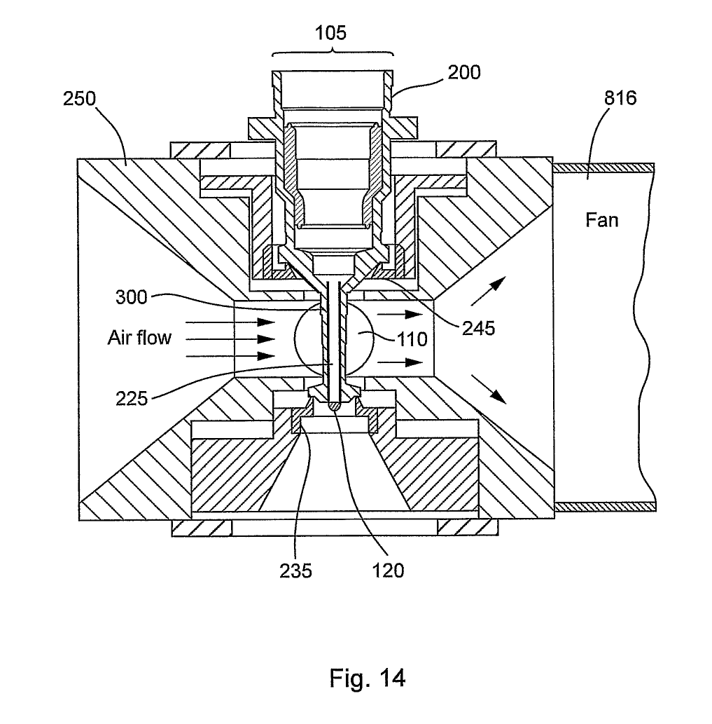

FIG. 14 shows a cross section of a chamber for use in the equipment of FIG. 1, the cross sectional view being perpendicular to the view shown in FIG. 2.

Referring to FIG. 1, a sample for fluorescence-based assay is delivered in known manner, via a sample delivery input 100, to a glass capillary coated in an electrically conductive polymer (“ECP”) to make a capillary assembly 105. The capillary assembly 105 is provided with a heating circuit having a drive current control 115 to deliver a drive current to heat the ECP and having an infrared-based thermopile 110 for dynamic feedback control to the drive current control 115. Excitation radiation 170 for use in exciting fluorescent probe activity is delivered in known manner to the capillary assembly 105 from a source 145, via a dichroic mirror 130 and a further lens 125. The capillary assembly 105 has a beaded end 120 through which it receives the excitation radiation 170 and delivers fluorescent output. Such arrangements are of known general type and an example is described in British patent GB 2334904.

Referring to FIG. 2, the capillary assembly 105 in practice comprises a generally tubular structure 200 made from ECP which receives a glass tube 225 in a stalk portion which protrudes downwards in use of the assembly 105. The stalk portion is open-ended and the end of the glass tube 225 is the beaded end 120 mentioned above which is exposed through the open end of the stalk for optical input/output to the tube 225.

Referring also to FIG. 3, overall the generally tubular structure 200 has a circular cross section which is wide in the upper part, for receiving samples, and narrow in the lower part, the stalk portion 300, where the tube 225 sits in use. Different structures may be found appropriate and, in a variation, there may for example be a tubular aluminium liner (not shown) between the stalk portion 300 of the ECP structure 200 and the tube 225.

Typical dimensions of the ECP structure 200 and the tube 225 might be for example a tube 225 that has a length 15 mm, an inside diameter of 1.33 mm and an outside diameter of 1.65 mm and an ECP stalk portion 300 that has an outside diameter of 3.5 mm. With a 20 mg sample, this means that heat applied to the tube 225 via the ECP material has less than 1 mm to travel to the centre of the sample and will take about three or four seconds.

The generally tubular structure 200 is supported in an aluminium chamber 250 which is generally cuboid when seen from outside. However, the chamber 250 has a more complex internal structure for receiving and supporting the tubular ECP structure 200, electrical connections 205 to it and a contactless heat sensor 110. The tubular ECP structure 200 is supported by a pair of copper collars 235, 245 spaced apart at either end of the stalk portion 300. The electrical connections 205 from the heating circuit 115, 110 are provided as wires to these copper collars 235, 245 which in turn are in direct contact with the ECP material and thus deliver drive current to it in the region of the stalk portion 300. The heat sensor 110 is mounted to one side of the stalk portion 300 of the ECP tubular structure 200 and thus receives infrared radiation from it. However, it also inevitably receives radiation from internal surfaces of the chamber 250 and other structures.

The heat sensor 110 is a thermopile or bolometer of known type, these being commercially available from suppliers such as General Electric (“GE”) or Calex Electronics Ltd.

The detailed construction of the chamber 250 and the supporting structures is not critical to embodiments of the present invention and is partly dictated by other factors such as air flow but it can be seen that the heat sensor 110, in the assembled chamber 250, has a far more complex field of “view” than simply the ECP material surface of the capillary assembly 105. Not only does it receive radiation from aluminium surfaces as well as the ECP but there will also be reflections from the various surfaces, including for example the copper collars 235, 245. These different materials will also tend to act as heat sinks for condudted heat. The view the heat sensor 110 has of the stalk portion 300 of the capillary assembly 105 is also complicated in that it will tend to see the extremities of it at a different temperature from the nearer, central portion of the stalk. Complicated environments such as this have to be modelled using a combination of analytical, computational and experimental approaches to derive the algorithms required to relate sensor outputs to sample temperature.

Referring to FIG. 4, the environment is yet further complicated by the structure of the heat sensor 110 itself. It may for example be based on a semiconductor chip 400 supported on a wafer 405, the chip receiving radiation through a window in a frame 410.

Immediate factors that would have to be taken into account if the behaviour of the heat sensor were to be modelled in order to relate its output to the temperature of the ECP stalk are:

1. The surfaces involved:

ECP stalk

Cavity around ECP

Aluminium frame 410 around the aperture into the heat sensor 110

Heat sensor chip 400

Heat sensor wafer 405 or whatever else surrounds the chip 400

2. Characterisation of these surfaces by area, emissivity and temperature

3. Form factors in terms of the shapes of, the distances from and the viewing angle to the surfaces.

There are many further factors to take into account which are affected by temperature and will therefore change during a heating or cooling operation, such as:
chip behaviour
the difference in temperature between the aluminium surfaces and the chip 400 and its effect on emissivity and the thermal contact between the heat sensor 110 and the chamber 250
the difference in temperature between the wafer 405 and the chip 400 since radiation emitted by the wafer 405 will be reflected from the frame 410 back to the chip 400
a reduction in sensitivity when the ECP stalk is hotter than the chip 400

Over time, there are very likely to be still further factors to take into account, such as tarnishing and dust which will both affect emissivity.

The thermal properties of the system are well defined through experiment and modelling and may be in the form of an algorithm or look-up table stored in memory. Because the condition of the system may change with time, the calibration settings may need to be adjusted.

The temperature of the ECP and container need to be controlled through heating and cooling cycles to affect PCR. Rapid temperatures changes are needed for fast cycling in order to keep assay times short, so the thermal mass is kept low to allow these rapid changes to occur. The system is therefore responsive to induced perturbation.

FIG. 8A is a schematic illustration showing the ECP coated capillary assembly 105, contactless heat sensor 110 and temperature control circuit 800. In normal use, emissions from the ECP induce a signal from the heat sensor that is fed to a control circuit. The signal is converted into a temperature reading (for example, using an algorithm or look-up table) and the difference between the required temperature and the measured temperature is calculated. The current to the heater or the cooling fan (not shown) needed to change the system from the measured temperature to the required temperature is then applied.

The conversion of the signal to a temperature reading requires a calibration of the system. The thermal properties of the system are well defined through experiment and modelling and thus can be used to predict the temperature of the ECP when a drive current is applied. However, as the condition of the system may change with time, the calibration settings may need to be adjusted. This can most conveniently be effected using the system itself where the well defined thermal properties of the ECP and capillary assembly, with a defined electrical pulse can be used to deliver a known temperature shift.

Other temperature sensors in the system such as thermistors or thermocouples that are not subject to aging effects can be used to provide an absolute setting.

FIG. 8B shows the feedback control cycle on the system shown in FIG. 1. In normal use, the feedback control cycle starts with the infra-red emissions from the ECP (A). These induce a signal from the heat sensor (B) that is fed to the temperature control circuit where the difference between the required temperature and the measured temperature is calculated. The heating current (or cooling airflow) needed to change the temperature to the required temperature is determined and this is then applied to the ECP (C).

For calibration, the sequence is altered, and a calibration feedback control cycle is illustrated in FIG. 8C. The control circuit sends a defined current pulse (D) to the ECP. The increased temperature of the ECP results in a change in its emissions (E) which affect the heat sensor. The response (F) of the heat sensor to the heated pulse is communicated to the temperature control circuit. The thermal properties of the ECP and capillary assembly are well defined so the current pulse (D) has a predictable effect on the temperature of the heater. Comparison of the predicted signal with (F) the received signal can thus be used to calibrate the signals from the sensor.

A more detailed description of the calibration will now be given, referring to FIG. 5. This is an embodiment of the invention which can take all these factors into account by calibrating in situ comprising a temperature control circuit 505 which incorporates both a drive current control 115 and a heat sensor calibrator 500, together with a data store 510 for storing drive sequences. The basis of the calibration is the principle that the temperature of the ECP stalk 300 is relatively simple to model because it has low thermal mass. The heat sensor calibrator 500 is provided by a software process which takes heat sensor readings as input, either directly from the heat sensor as indicated in FIG. 5, or via a data store 510, and correlates the readings with calculated temperature values for the ECP stalk 300. In order to correlate the readings accurately, the heat sensor calibrator 500 runs a known test pattern in the drive current to the stalk 300, such as a series of pulses. The correlated readings can simply be output by the calibrator 500 to another software system or to data storage for subsequent use but more usefully might be applied directly to subsequent drive currents to compensate for any drift in heat sensor outputs.

The heat sensor calibrator 500 can be described as a drive current controller for applying the test sequence, a detector for detecting an output of the heat sensor and a correlator for correlating heat sensor outputs with features of the test sequence. The drive current controller is a signal output to the drive current control 115 itself, for instance selecting a drive sequence that is suitable for calibration. Preferably, the signal output includes the calibration drive sequence itself and the drive current controller may read this from the data store 510. The detector for detecting an output of the heat sensor may be simply an input that is only activated to detect and store readings when the heat sensor calibrator 500 is being run. The correlator for correlating heat sensor outputs with features of the test sequence may simply apply a filter to the readings to select only those applicable to a time window when a calibration pulse is present in the drive sequence. These selected readings may then be compared to the calculated readings for those time windows.

It might be noted that FIG. 5 shows drive current being applied to the ECP stalk 300 by electrical connectors 515 such as wires. However, the drive current could equally be applied inductively, thus reducing physical connections to the stalk 300.

To apply the correlated readings directly, the calibrator 500 adjusts the relationship between subsequent heat sensor readings and the drive current supplied to the ECP stalk 300. It can do this by converting the heat sensor readings before they are received by the drive current control 115 or by changing the response of the drive current control 115 to the subsequent readings. The former is generally the easier option since there is no requirement for a change in the operation of the drive current control 115. In embodiments of the invention using the former approach, the heat sensor readings will be received by the calibrator 500 instead of by the drive current control 115. The input of the drive current control 115 will instead be connected to the calibrator 500 and receive adjusted heat sensor readings which the calibrator has adjusted on the basis of stored or fresh calibration sequence results.

Referring to FIG. 6, in a normal PCR operation a sample is repeatedly driven through a heating/cooling cycle. The heating is provided by the ECP stalk 300 as described above while cooling is provided using a fan to blow air through the chamber 250. The chamber 250 has an opening through it for this purpose, orthogonal to the direction of the heat sensor 110 as shown in FIG. 2. The heating stage is done for instance at constant drive current 605 and the ECP will heat at a constant rate 600. A typical ECP stalk 300 weighs about 20 mg and the sample another 20 mg or so. A drive current at 1 Watt delivers about 1 Joule per second energy. One calorie of energy heats 1 g through 1° C. and one calorie is 4.18 Joules. Thus a drive current of 4 W will heat a sample of 0.1 ml through 10° C. FIG. 6 shows the calculated temperature of the ECP stalk 300 in response to a drive current 605 of 4 W for 10 seconds.

In a usual PCR operation, this heating stage would be followed by a cooling stage and the two stages repeated several times. The drive current is switched on and off in response to readings of the heat sensor 110 which is monitoring the temperature of the ECP stalk 300. However, as discussed above, problems can arise if the readings of the heat sensor 110 give a misleading temperature for the ECP stalk 300.

Referring to FIG. 7, the readings of the heat sensor 110 may generate a curve 700 which in fact deviates from the actual (calculated) temperature 600 of the ECP stalk 300. The deviation may be constant but is more likely to be affected by the instantaneous temperature of the stalk 300, for instance being greater at higher temperatures as shown. This can be detected by putting a sample of reagent in the capillary assembly 105 and using a test drive current sequence to produce a known temperature in the ECP stalk 300, for instance a calculated temperature 600, and comparing the temperature curve 700 indicated by the heat sensor 110. This allows both a one-off calibration and a calibration that detects changes over time.

In light of the thermal mass of an ECP stalk 300, a typical test drive current sequence might incorporate 4 Watt pulses 705, 710 superimposed on a steady drive current for producing a ramped increase in temperature of the sample. The pulses might be superimposed at low sample temperature and high, within the normal working range of the apparatus. Thus as shown in FIG. 7, pulses 705, 710 are used before the steady drive current is applied and after the temperature of the sample has passed 120° C.

In an example of an ECP capillary tube assembly 105 having an aluminium liner, the heated region may weigh as follows:
ECP material weighs 182 mg empty
ECP material weighs 207 mg with a 25 ml aqueous sample added
the glass capillary weighs 27 mg.
the aluminium sleeve around the capillary weighs approximately 46 mg

The composition of the materials being heated is thus 25 mg aqueous sample, 46 mg aluminium, 27 mg glass and 109 mg ECP. The specific heat capacities are:

| | | |
|---|---|---|
| Water | 1.0 | Cal/g/K |
| Al | 0.215 | Cal/g/K |
| Glass | 0.2 | Cal/g/K |
| ECP | 0.2 | Cal/g/K (estimated) |

Therefore it takes 0.025+0.0099+0.0054+0.022=0.062 calories to heat the assembly by 1 degree, which is 0.26 Joules. So it takes 2.6 Joules to heat it by 10 degrees. For 10 degrees per second, which is a typical ramp rate, this requires 2.6 Watts of power to be applied through the ECP material. With losses, 4 Watts is about right for the heating power used and calibration pulses will need to be in this range, say from 0.1 to 10 Watts.

The duration of the calibration pulses 705, 710 is chosen so that the heat sensor 110 sees the immediate response of the ECP material, before heat is conducted into the sample to any great extent. Hence the duration of the pulses 705, 710 is significantly less than three or four seconds, for instance less than a second and more preferably no more than 500 milliseconds (“msecs”).

As mentioned above, ECP is not the only suitable material for use as described above in providing heat to a biochemical sample. Other electrically resistive, conductive materials could well be substituted, such as true conductors, doped polyacetylene or polyaniline, or inorganic materials such as indium tin oxide. However, it is preferable that the material should be optically opaque. More than one different material may be used and the glass tube described above is also optional.

The apparatus and method may also be used to determine whether the thermal control (i.e. the heat sensor and the drive current) is functioning correctly.

In this embodiment, the temperature control circuit of FIG. 8A is used. As shown in FIG. 8C, the control circuit sends a defined current pulse to the heater. The increased temperature of the heater results in a change in its emission which affect the sensor. The response of the temperature sensor to the heating pulse is communicated to the temperature control circuit.

FIG. 9 illustrates the sequence events, the first three steps being equivalent to those illustrated in FIG. 8C. In a first step 802 a sequence of defined current pulses is applied to the ECP. The heat sensor detects the temperature of the ECP 804. The response of the sensor is input to the temperature control circuit. In the temperature control circuit, the received signal is compared with the predicted signal 806 and it is determined whether the received signal is within a predefined range 808. If it is, then the thermal control is functioning. If not, further investigation is required.

The first check on the occurrence of abnormal responses is to determine the response to a pulse of cooling air. The effect is independent of the heating system and can therefore be used to ascertain whether unpredicted signals received in the test sequence result from changes in the temperature measuring system or from deviations in the thermal mass of the sample.

The method and apparatus of this invention can therefore also be used to determine information about the sample within the container. As discussed in the description of a previous embodiment, the calories required to heat the assembly by 1 degree were calculated. The water in the aqueous sample required 0.025 Calories, whilst the capillary assembly required 0.0373 Calories. Thus 40% of the energy to heat the assembly by 1 degree is required to heat the water. This is because the specific heat capacity of water is significantly higher than any of the components in the capillary assembly.

As such a significant amount of energy is required to heat the sample, the method and apparatus of this invention can be used to determine whether any sample is present at all. If no sample is present, the capillary assembly will heat to a higher temperature in reaction to the defined current pulse and thus a higher temperature will be detected by the heat sensor.

FIG. 10 illustrates the steps for determining whether a sample is present, the first few steps being the same as described in FIG. 9. As before, the temperature control circuit is used to produce a defined test sequence of current pulse to the ECP as illustrated in FIG. 8C. The emitted heat from the ECP is detected by the heat sensor and its output is received by the temperature control circuit.

The measured temperature is compared with the predicted temperature if a sample present. The difference between the measured and predicted temperatures is determined 810 and this is used to determine the measured temperature is within a predefined range of the predicted temperature 812. If it is, the sample is present. Otherwise the sample is absent or partially absent.

The specific heat capacity of different aqueous solutions will vary and this technique is sufficiently accurate to differentiate between, thus enabling it to be determined whether the correct sample has been put into the capillary assembly. In this case a table containing the predicted temperature for a predefined volume of different samples is saved in memory, either in the temperature control circuitry or in a separate location. The measured temperature can thus be compared with data from the look-up table to determine the contents of the sample.

FIG. 11 is a graph illustrating the effect of a heating pulse on different samples. The heating pulse 814 is applied for a predetermined time for three different samples (oil, water and no sample). The results show quantitative differences in the derived temperature according to the contents of the capillary assembly.

The chamber which supports the capillary assembly also includes a fan to blow air and thus cool the capillary assembly. The fan can be controlled to blow air in a test sequence, for example one or more pulses. As before, the test sequence is applied, the temperature of the ECP is measured and the measured temperature output to the temperature control circuit. The responses, especially in combination with the use of heating pulse test sequences as described above, can be used to ascertain the status of temperature measurement system and the functioning of the cooling fan, the accuracy of the check being improved by the independency of the heating and cooling test sequences.

FIGS. 12 and 13 are graphs illustrating the change in temperature of the ECP measured by the heat sensor an application of a heat pulse and cooling pulse respectively.

In FIG. 12, the capillary assembly has been filled with water and stabilised at 50° C. A heating pulse (square symbols) is applied between from 33.75 to 38.75 seconds. The temperature is controlled when the derived internal temperature (diamond symbols) reaches 90° C.

In FIG. 13, the capillary assembly has been filled with water and stabilised at 90° C. A cooling pulse is applied between 69 and 87 seconds. The temperature is controlled after the derived internal temperature falls below 50° C.

The forms of the curves in the two graphs are different. Different models are used to determine the predicted temperature of the ECP in response to the application of a heating or cooling pulse. The difference is due to the different manner of heating and cooling. Heating by applying a current to the ECP is a very direct way of heating the sample. However when cooling with an air flow, the ECP coating the capillary assembly must first be cooled before causing the capillary assembly and then sample to be cooled.

As different models are used to describe heating and cooling, the methods described in the embodiments above can be further improved by carrying out the method twice, once with heating pulses and again with cooling pulses.

FIG. 14 is a cross-sectional view of the chamber supporting the capillary assembly, taken from a view perpendicular to that shown in FIG. 2. Features identical to those in FIG. 2 are shown with the same reference numerals. As in FIG. 2, the capillary assembly 105 is shown located in the chamber. The heat sensor 110 can be seen end on, behind the stalk portion 300 of the capillary assembly. A fan 816 is provided to draw air through the chamber, as shown by the arrows.

1. An apparatus for applying monitored temperature changes to samples held in containers, the apparatus comprising:
i) a chamber for holding one or more containers;
ii) an electrically conductive material for mounting in association with a container in the chamber so as to heat the container in use;
iii) one or more contactless heat sensors for measuring a temperature of at least a portion of the electrically conductive material;
iv) a drive current source for applying a drive current to the electrically conductive material, when in the chamber, so as to change its temperature; and
v) a control circuit adapted to control the drive current to follow a test sequence for use in calibrating the one or more contactless heat sensors, testing thermal control or determining a contents of the container.

2. The apparatus according to claim 1 wherein the control circuit is connected to an output of the heat sensor.

3. The apparatus according to claim 1 wherein the control circuit calibrates a response of at least one heat sensor to a temperature of the electrically conductive material and wherein the control circuit is provided with a data store for storing data against which to calibrate said response.

4. The apparatus according to claim 1, further comprising a feedback circuit for controlling the drive current to the electrically conductive material in accordance with an output of the heat sensor, during use of the apparatus.

5. The apparatus according to claim 4, wherein a calibrator is connected to the feedback circuit for controlling the drive current in accordance with an output of the heat sensor after calibration.

6. The apparatus according to claim 1, wherein the electrically conductive material is provided as a sleeve or other cover which can be brought into close contact with the container.

7. The apparatus according to claim 1 wherein the apparatus comprises a heat treatment apparatus for biochemical samples and the container is a biochemical sample container.

8. The apparatus according to claim 7 for use in polymerase chain reaction processes.

9. The apparatus according to claim 1 wherein the electrically conductive material comprises a polymer.

10. The apparatus according to claim 1 wherein a cross section of an outermost surface of the container in a region of the electrically conductive material has a minimum dimension of not more than 5 mm.

11. The apparatus according to claim 1 wherein a cross section of an outermost surface of the container in the region of the electrically conductive material has a minimum dimension of less than 3 mm.

12. The apparatus according to claim 1 wherein a mass of the electrically conductive material subject to the drive current is not more than 0.5 g.

13. The apparatus according to claim 1 wherein a mass of the electrically conductive material subject to the drive current is not more than 0.25 g.

14. The apparatus according to claim 1, wherein the test sequence comprises two or more pulses of drive current superimposed on a drive current sequence for raising a temperature of the container, such that the two or more pulses are applied to the electrically conductive material at different respective temperatures thereof.

15. The apparatus according to claim 1 wherein the test sequence comprises at least one pulse of drive current of not more than a one second duration.

16. The apparatus according to claim 13 wherein the pulse or pulses have a duration of not more than 500 msecs.

17. The apparatus according to claim 1 wherein the test sequence comprises at least one pulse of a size to change a power level of the drive current in a range from 0.1 to 10 Watts.

18. The apparatus according to claim 1 further comprising a source of cool air so as to cool the container in use, a drive current source for applying a drive current to the source of cool air, when in the chamber, so as to change its temperature; and a control circuit adapted to control the drive current to follow a test sequence.

19. A method of heat treating a sample in a container, the container comprising at least in part at least one electrically conductive wall, the method using one or more contactless heat sensors to sense the temperature of said wall, which method comprises the steps of:
i) applying a drive current to the electrically conductive wall;
ii) varying the drive current according to a predetermined test sequence;
iii) monitoring a response of at least one of the one or more heat sensors to the test sequence;
iv) comparing the monitored response to a predicted temperature of the electrically conductive wall; and
v) using the comparison for calibrating the one or more contactless heat sensors, testing thermal control or determining a contents of the container.

20. The method according to claim 19 comprising the additional step of heat treating the sample, using the monitored response of the heat sensor in a feedback loop to control the drive current to heat the electrically conductive walls.
